(19) United States
(12) Patent Application Publication
(10) Pub. No.: US 2024/0023803 A1
(43) Pub. Date: Jan. 25, 2024

(54) MEDICAL INSTRUMENT SET AND METHOD OF USING THE SAME

(71) Applicant: KANEKA CORPORATION, Osaka-shi, Osaka (JP)

(72) Inventors: Kotaro MIYAKE, Suita-shi, Osaka (JP); Shintaro OSUMI, Settsu-shi, Osaka (JP); Keisuke MATSUSHITA, Minato-ku, Tokyo (JP)

(73) Assignee: KANEKA CORPORATION, Osaka-shi, Osaka (JP)

(21) Appl. No.: 17/768,382

(22) PCT Filed: Sep. 25, 2020
(86) PCT No.: PCT/JP2020/036321

§ 371 Date: Apr. 12, 2022

(30) Foreign Application Priority Data
Oct. 16, 2019 (JP) 2019-189684

(51) Int. Cl. A61B 1/267; A61B 1/00; A61B 1/018; A61B 1/01; A61M 25/10

(52) U.S. Cl. CPC A61B 1/2676; A61B 1/00082; A61B 1/00131; A61B 1/018; A61B 1/01; A61M 25/10; A61M 2025/1079

(57) ABSTRACT

An object of the present invention is to provide a medical instrument set facilitating safe observation of a peripheral bronchus portion, and a method of using the same. The medical instrument set includes a bronchoscope including a sheath having a longitudinal axis direction, the sheath having a first lumen and a second lumen each extending in the longitudinal axis direction, an endoscopic camera disposed in the first lumen, and a balloon catheter including a shaft inserted to the second lumen to be shiftable in the longitudinal axis direction and a balloon provided at a distal portion of the shaft; and a probe having a distal end positioned distally beyond a distal end of the balloon, the probe configured to detect a peripheral bronchus portion.

### TECHNICAL FIELD

The present invention relates to a medical instrument set facilitating safe observation of a peripheral bronchus portion, and a method of using the same.

### BACKGROUND ART

There have been so far known various lung diseases such as chronic obstructive pulmonary disease (COPD), chronic bronchitis, and lung cancer. Various examinations and treatments have been executed for these lung diseases, and there have been known various examination methods, treatment instruments, and the like.

For example, Patent Document 1 discloses an endoscopic catheter including a shaft, an image detector, and guide wire, as well as a balloon portion provided at a distal end of a body of the shaft. Patent Document 1 further describes that the endoscopic catheter can be moved forward to a desired position in an endolumen in a living body such as a bronchus for image diagnosis.

### RELATED ART DOCUMENTS

### Patent Documents

Patent Document 1: JP-A-2016-182302

### SUMMARY OF THE INVENTION

### Problems to be Solved by the Invention

Lung cancer sometimes occurs at a peripheral bronchus portion. However, conventional techniques have difficulty in inserting a bronchoscope to the peripheral bronchus portion, which is extremely thin, for observation with use of an endoscopic camera. For example, the endoscopic catheter according to Patent Document 1 is difficult to be inserted to a peripheral bronchus portion due to large insertion resistance because a bronchoscope is inserted to a bronchus with a balloon being pressurized, and due to a large diameter of the balloon that is provided at an outer side surface of the endoscopic catheter. Furthermore, the peripheral bronchus portion has a terminal adjacent to a visceral pleura. Damage to the visceral pleura may lead to pneumothorax with a perforated lung tissue, so that safety is needed upon observation.

The present invention has been achieved in view of the above circumstances, and an object thereof relates to a medical instrument set facilitating safe observation of a peripheral bronchus portion, and a method of using the same.

### Solutions to the Problems

The present invention provides a medical instrument set that successfully solves the above problems and is configured as follows.
[1] A medical instrument set including:
a bronchoscope including
a sheath having a longitudinal axis direction, the sheath having a first lumen and a second lumen each extending in the longitudinal axis direction,
an endoscopic camera disposed in the first lumen, and
a balloon catheter including a shaft inserted to the second lumen to be shiftable in the longitudinal axis direction and a balloon provided at a distal portion of the shaft; and
a probe having a distal end positioned distally beyond a distal end of the balloon, the probe configured to detect a peripheral bronchus portion.

The bronchoscope includes the balloon catheter inserted to the second lumen of the sheath to be shiftable in the longitudinal axis direction, thus the user can distally extrude the balloon from the second lumen of the sheath, expand and contract the balloon, and then pull back the balloon into the second lumen. In this configuration, the peripheral bronchus can be expanded by the balloon and the bronchoscope can then be inserted to the peripheral bronchus with the balloon being contracted, thereby reducing the insertion resistance. Furthermore, forcible and excessive insertion of the balloon catheter may lead to damage to a visceral pleura adjacent to a terminal of a peripheral bronchus portion. Adopting the probe configured to detect a peripheral bronchus portion enables positional detection of the terminal of the peripheral bronchus portion, thus, facilitating avoidance of damage to the visceral pleura.

Further, preferred aspects of the medical instrument set according to the present invention are as follows [2] to [14].
[2] The medical instrument set according to above [1], wherein the probe is inserted to the second lumen of the bronchoscope.
[3] The medical instrument set according to above [1] or [2], wherein the shaft and the probe each include a radiopaque portion.
[4] The medical instrument set according to above [3], wherein the radiopaque portion of the probe is positioned distally beyond the radiopaque portion of the shaft.
[5] The medical instrument set according to above [3] or [4], wherein the shaft includes the radiopaque portion positioned proximally beyond a proximal end of an unfixed portion of the balloon, the unfixed portion not being fixed to the shaft.
[6] The medical instrument set according to any one of above [3] to [5], wherein the radiopaque portion is a radiopaque marker.
[7] The medical instrument set according to any one of above [1] to [6], wherein the balloon has a distance (mm) in a longitudinal axis direction of the shaft from a distal end of an unfixed portion not fixed to the shaft to the distal end of the probe, the distance being 0.5 times or more an outer diameter (mm) of the balloon provided therein with fluid at a center in the longitudinal axis direction of the shaft.
[8] The medical instrument set according to any one of above [1] to [7], further including a guide wire provided in the second lumen.
[9] The medical instrument set according to any one of above [1] to [8], wherein the probe has a distal end portion including a curved portion having a curvature radius of 0.1 mm or more and 10 mm or less in a sectional view in a longitudinal axis direction.
[10] The medical instrument set according to any one of above [1] to [9], wherein the probe contains a resin.
[11] The medical instrument set according to any one of above [1] to [10], wherein the probe contains a metal.
[12] The medical instrument set according to any one of above [1] to [11], wherein the probe has a lumen having a longitudinal axis direction.
[13] The medical instrument set according to any one of above [1] to [12], wherein the probe includes a columnar portion having an outer diameter (mm) smaller than an outer diameter of the shaft at a proximal end of the balloon.
[14] The medical instrument set according to any one of above [1] to [13], wherein the probe includes a columnar portion and a spherical portion provided at a distal end of the columnar portion, and the spherical portion has an outer diameter larger than an outer diameter of the columnar portion.

The present invention further includes a method of using the medical instrument set as in below.
[15] A method of using the medical instrument set according to any one of above [1] to [14], the method including inserting the probe to the second lumen, extruding a distal end portion of the probe from a distal end of the second lumen, and then extruding the balloon from the distal end of the second lumen.

### Effects of the Invention

According to the present invention, the configurations described above enable provision of a medical instrument set facilitating safe observation of a peripheral bronchus portion, as well as a method of using the same.

### BRIEF DESCRIPTION OF THE DRAWINGS

FIG. 1 is a perspective view of a medical instrument set according to an embodiment of the present invention.

FIG. 2 is a side view (partially sectional view) depicting a method of using the medical instrument set according to the embodiment of the present invention.

FIG. 3 is a side view (partially sectional view) depicting the method of using the medical instrument set according to the embodiment of the present invention.

FIG. 4 is a side view depicting the method of using the medical instrument set according to the embodiment of the present invention.

FIG. 5 is a sectional view in a longitudinal axis direction of a balloon catheter in a region A illustrated in FIG. 3.

FIG. 6 is a sectional view in the longitudinal axis direction of an exemplary probe according to the embodiment of the present invention.

FIG. 7 is a sectional view in the longitudinal axis direction of a different exemplary probe according to the embodiment of the present invention.

FIG. 8 is a sectional view in the longitudinal axis direction of a different exemplary probe according to the embodiment of the present invention.

FIG. 9 is a sectional view in the longitudinal axis direction of a different exemplary probe according to the embodiment of the present invention.

FIG. 10 is a view depicting a different exemplary section in the longitudinal axis direction of the balloon catheter in the region A illustrated in FIG. 3.

FIG. 11 is a plan view of a distal end of a sheath included in a bronchoscope according to the embodiment of the present invention.

### MODE FOR CARRYING OUT THE INVENTION

More specific description will now be made to the following embodiment of the present invention. The present invention should not be limited by the following embodiment, but can obviously be implemented with appropriate modifications within a range applicable to purport of the above and below description. Such modifications will be all included in the technical scope of the present invention. Some of the drawings may not include reference signs of members for convenience, in which case the specification or any other appropriate drawing should be referred to. Various members in the drawings may not be depicted in actual sizes, because contribution to comprehension of the features of the present invention is prioritized in the drawings.

A medical instrument set according to the present invention includes: a bronchoscope including a sheath having a longitudinal axis direction, the sheath having a first lumen and a second lumen each extending in the longitudinal axis direction, an endoscopic camera disposed in the first lumen, and a balloon catheter including a shaft inserted to the second lumen to be shiftable in the longitudinal axis direction and a balloon provided at a distal portion of the shaft; and a probe having a distal end positioned distally beyond a distal end of the balloon, the probe configured to detect a peripheral bronchus portion.

In the medical instrument set according to the present invention, the bronchoscope includes the balloon catheter inserted to the second lumen of the sheath to be shiftable in the longitudinal axis direction, thus the user can distally extrude the balloon from the second lumen of the sheath, expand and contract the balloon, and then pull back the balloon into the second lumen. In this configuration, the peripheral bronchus can be expanded using the balloon and the bronchoscope can then be inserted to the peripheral bronchus with the balloon being contracted, thereby reducing the insertion resistance. Furthermore, adopting the probe configured to detect a peripheral bronchus portion when inserting the balloon catheter enables positional detection of a terminal of the peripheral bronchus portion, thus, facilitating avoidance of damage to a visceral pleura by the balloon catheter. The medical instrument set according to the present invention can thus achieve reduction in insertion resistance as well as easy avoidance of damage to the visceral pleura by the balloon catheter, to facilitate safe observation of the peripheral bronchus portion.

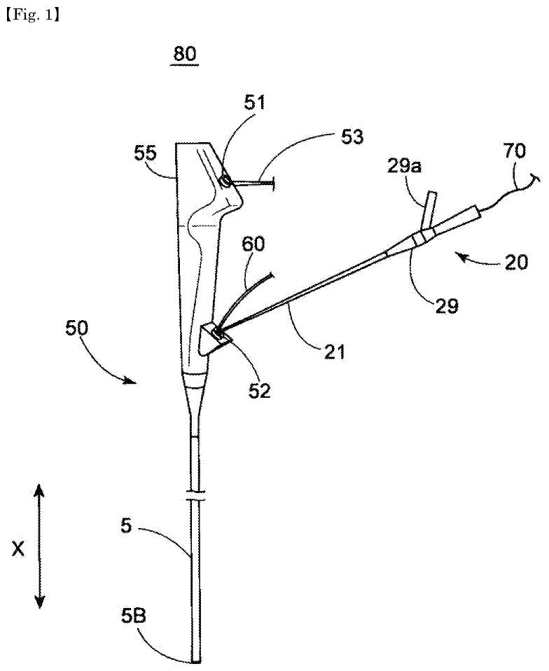

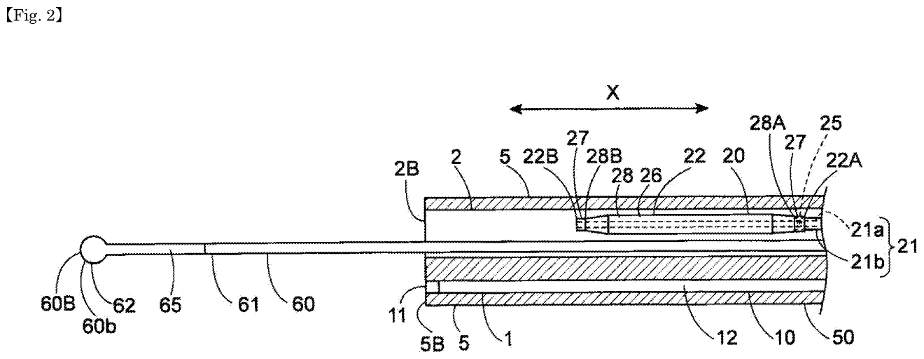

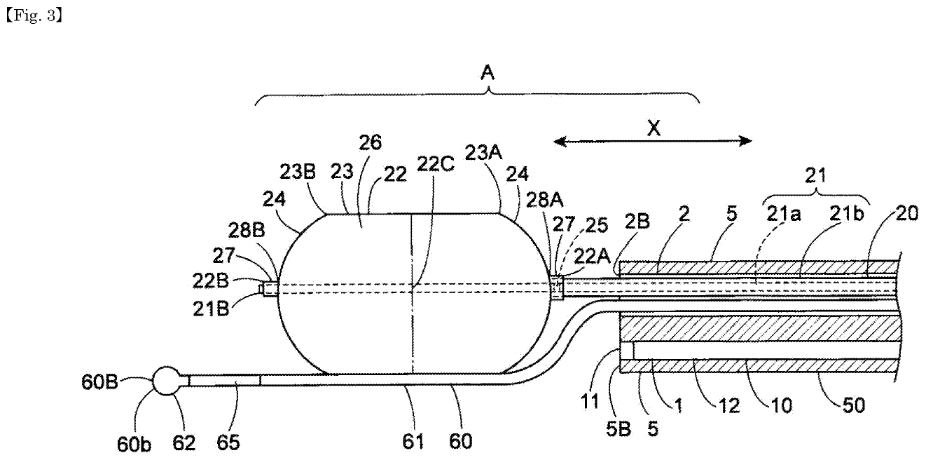

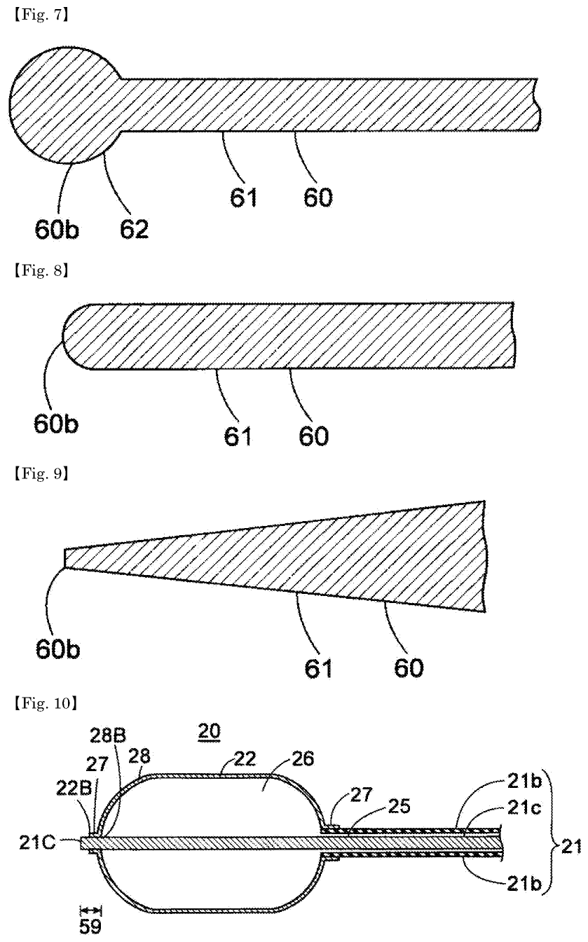

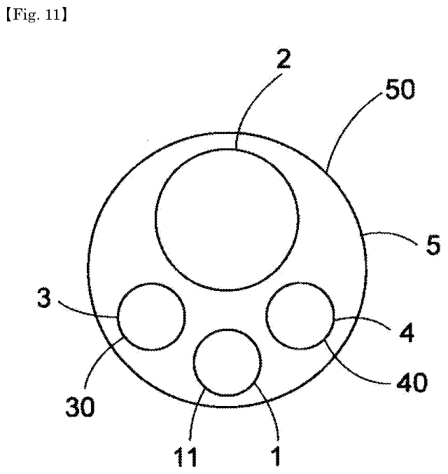

With reference to FIGS. 1 to 11, description is made hereinafter to a medical instrument set according to an embodiment of the present invention, and a method of using the same. FIG. 1 is a perspective view of the medical instrument set according to the embodiment of the present invention. FIGS. 2 to 4 are side views depicting the method of using the medical instrument set according to the embodiment of the present invention. FIGS. 2 and 3 include hatched portions indicating a section of a sheath. FIG. 3 further includes dashed lines indicating an outer diameter of a balloon at a center in a longitudinal axis direction of a shaft. FIG. 5 is a sectional view in a longitudinal axis direction of a balloon catheter in a region A illustrated in FIG. 3. FIG. 6 is a sectional view in the longitudinal axis direction of an exemplary probe according to the embodiment of the present invention. FIGS. 7 to 9 are sectional views in the longitudinal axis direction of a different exemplary probe according to the embodiment of the present invention. FIG. 10 is a view depicting a different exemplary section in the longitudinal axis direction of the balloon catheter in the region A illustrated in FIG. 3. FIG. 11 is a plan view of a distal end of the sheath included in a bronchoscope according to the embodiment of the present invention.

FIGS. 1 and 2 depict a medical instrument set 80 including a bronchoscope 50 and a probe 60. The bronchoscope 50 includes a sheath 5 having a longitudinal axis direction X, the sheath having a first lumen 1 and a second lumen 2 each extending in the longitudinal axis direction X. The sheath 5 has an outer diameter that is preferably 6 mm or less and is more preferably 3 mm or less. The sheath 5 thus configured can be easily inserted to a peripheral bronchus having an inner diameter of 2 mm or less. Meanwhile, the outer diameter of the sheath 5 has a lower limit that is preferably 1 mm or more and is more preferably 2 mm or more in consideration of a size of an endoscopic camera 10 to be described later, and the like. The sheath 5 is made of a material, examples of which include a polyamide resin, a polyester resin, a polyurethane resin, a polyolefin resin, a vinyl chloride resin, a silicone resin, a fluororesin, an epoxy resin, and natural rubber. The sheath 5 may be made of only one of these materials, or two or more of these materials.

As depicted in FIG. 2, the first lumen 1 is provided therein with the endoscopic camera 10. The endoscopic camera 10 is preferably fixed to the first lumen 1 so as not to move in the longitudinal axis direction X. The endoscopic camera 10 is not particularly limited, and exemplarily includes an object lens 11 and an image transmitter 12. Examples of the image transmitter 12 include image guide fiber constituted by optical fiber, and a relay lens constituted by a large number of lenses. The endoscopic camera acquires image information that is transmitted to a display device via a connector portion 53 of the bronchoscope 50 depicted in FIG. 1 or the like, and the display device displays the image information.

As depicted in FIG. 2, a balloon catheter 20, which includes a shaft 21 and a balloon 22 provided at a distal portion of the shaft 21, is inserted to the second lumen 2 of the sheath 5 so as to be shiftable in the longitudinal axis direction X. Specifically, the balloon catheter 20 is inserted to the second lumen 2 of the sheath 5 via an insertion hole 52 of the bronchoscope depicted in FIG. 1, and the balloon catheter 20 is not fixed so as to be shiftable in the longitudinal axis direction X. Furthermore, the balloon 22 includes a pressurization lumen 26 to be provided with fluid to receive positive pressure. In the bronchoscope 50 thus configured, a balloon 22 inserted to the second lumen 2 can be distally pushed from the second lumen 2 to expand the balloon 22 as depicted in FIG. 3, and the balloon 22 can be contracted and then be pulled back into the second lumen 2. Furthermore, the sheath 5 can be inserted to a peripheral bronchus being expanded, in a state where the balloon 22 is accommodated in the second lumen 2.

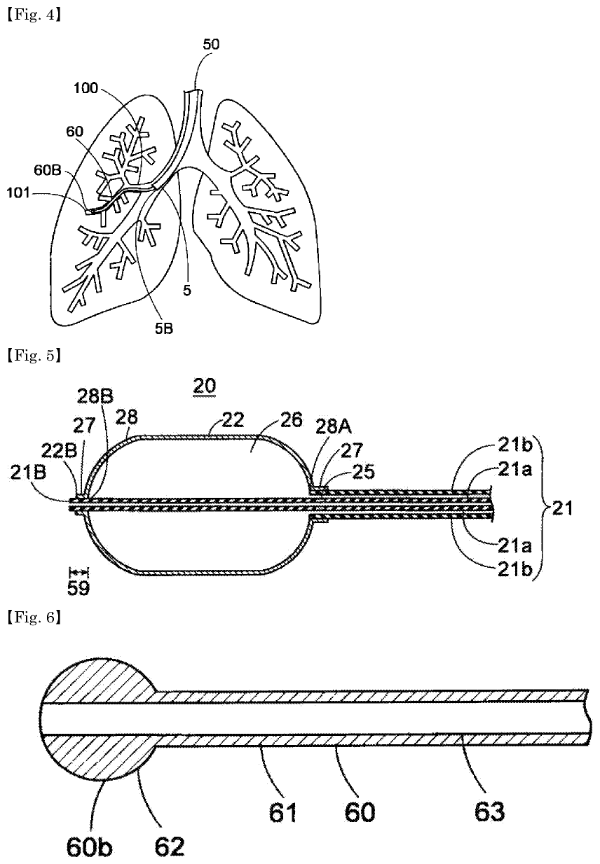

As depicted in FIGS. 2 and 4, the probe 60 has a distal end 60B positioned distally beyond a distal end 22B of the balloon 22, and is configured to detect a peripheral bronchus portion 101. The probe 60 is configured to detect the peripheral bronchus portion 101 by means of a texture of the peripheral bronchus portion 101 in contact with the distal end and is not particularly limited in terms of length in a longitudinal axis direction and thickness. As depicted in FIGS. 2 and 4, the probe 60 precedes a balloon catheter 20 to detect the peripheral bronchus portion 101, so that distance to the peripheral bronchus portion 101 can be grasped in accordance with length of an inserted portion of the probe 60 or the like. This enables easy avoidance of damage to a visceral pleura due to excessive pushing of the balloon catheter 20.

The probe 60 is preferably inserted to the second lumen 2 of the bronchoscope 50. The probe 60 is inserted to the identical lumen that receives the balloon catheter 20 without need to provide another lumen to receive the probe 60, which facilitates reduction in outer diameter of the sheath 5.

Each of a shaft 21 and the probe 60 preferably includes a radiopaque portion. The radiopaque portion contains a radiopaque substance. As depicted in FIGS. 2, 3, 5, and the like, the shaft 21 includes an inner tube 21a having a radiopaque portion 25, and the probe 60 has a radiopaque portion 65. The probe 60 is configured to detect the peripheral bronchus portion 101 in accordance with a texture upon insertion. Optional provision of the radiopaque portion 65 facilitates positionally grasping the probe 60 by means of a fluoroscopic X-ray image, computed tomography, or the like, and thus facilitates detection of the peripheral bronchus portion 101. Similarly, optional provision of the radiopaque portion 25 at the shaft 21 facilitates positionally grasping the shaft 21 by means of a fluoroscopic X-ray image, computed tomography, or the like. The radiopaque portion 25 may not necessarily be provided to only part of the shaft 21, but may alternatively extend from a proximal end to a distal end of the shaft 21. The radiopaque portion 65 may not necessarily be provided to only part of the probe 60, but may alternatively extend from a proximal end to the distal end of the probe 60.

Examples of the radiopaque substance include at least one selected from a group consisting of lead, barium, iodine, tungsten, aurum, platinum, iridium, a platinum-iridium alloy, stainless steel, titanium, a cobalt-chromium alloy, palladium, and tantalum.

As depicted in FIGS. 2 and 3, the radiopaque portion 65 of the probe 60 is preferably positioned distally beyond the radiopaque portion 25 of the shaft 21. This disposition facilitates grasping positional relation between the probe 60 and the shaft 21 in a bronchus 100, which facilitates avoidance of damage to a visceral pleura by the balloon catheter 20 that is pushed distally beyond the distal end B of the probe 60.

The shaft 21 includes the radiopaque portion 25, whereas the probe may not include the radiopaque portion 65. The probe 60 not including the radiopaque portion 65 can detect the peripheral bronchus portion 101 in accordance with a texture upon insertion. Furthermore, distance to the peripheral bronchus portion 101 is grasped in accordance with length of an inserted portion of the probe 60 or the like, to facilitate avoidance of damage to a visceral pleura due to excessive pushing of the balloon catheter 20.

As depicted in FIGS. 2, 3, and 5, the shaft 21 preferably includes the radiopaque portion 25 positioned proximally beyond a proximal end 28A of an unfixed portion 28 of the balloon 22, the unfixed portion not being fixed to the shaft 21. This disposition facilitates grasping a position of the balloon 22 in a body.

The shaft 21 preferably includes the radiopaque portion 25 in at least one of a distal fixed portion 27 or a proximal fixed portion 27, where the balloon 22 is fixed. The radiopaque portion 25 is more preferably provided at each of the distal fixed portion 27 and the proximal fixed portion 27. This disposition facilitates grasping a position of the balloon 22 in a body.

The shaft 21 preferably includes the radiopaque portion 25 in a region within 20 mm from a center 22C of the balloon 22 in a longitudinal axis direction of the shaft 21, more preferably includes the radiopaque portion 25 in a region within 15 mm therefrom, and further preferably includes the radiopaque portion 25 in a region within 10 mm therefrom. This disposition facilitates grasping a site of the peripheral bronchus portion 101 expanded by the balloon 22.

The shaft 21 may not necessarily include only one radiopaque portion 25, but may alternatively include two or more radiopaque portions 25. In an exemplary case where the balloon 22 includes a straight tube portion 23, the shaft 21 preferably includes the radiopaque portions 25 at positions corresponding to a proximal end 23A and a distal end 23B of the straight tube portion 23. This configuration facilitates grasping a site of the peripheral bronchus portion 101 expanded by the straight tube portion 23.

The probe 60 may not necessarily include only one radiopaque portion 65, but may alternatively include two or more radiopaque portions 65. The radiopaque portion 65 is preferably provided in a region within 20 mm in the longitudinal axis direction from the distal end 60B of the probe 60, and is more preferably provided in a region within 15 mm therefrom. This disposition facilitates grasping a position of the distal end 60B of the probe 60. Meanwhile, in a case where the radiopaque portion 65 is a radiopaque marker to be described later, the radiopaque portion 65 is preferably provided in a region distant by 0.5 mm or more in the longitudinal axis direction from the distal end 60B of the probe 60, and is more preferably provided in a region distant by 1.0 mm or more therefrom. This disposition facilitates avoidance of damage to the peripheral bronchus portion 101 by the radiopaque marker. In a case where the probe 60 includes a spherical portion 62 to be described later, the radiopaque marker is preferably provided adjacent to a proximal end of the spherical portion 62.

At least one of the radiopaque portion 25 or the radiopaque portion 65 may be the radiopaque marker. The radiopaque marker is not particularly limited in terms of its shape, and examples of the shape include a ring shape and a spiral shape. The radiopaque marker is made of a material exemplified by the radiopaque substance. The radiopaque marker has a longitudinal axis length that is preferably 1.0 mm or more and more preferably 1.5 mm or more, and is preferably 20 mm or less and more preferably 15 mm or less.

Alternatively, each of the radiopaque portion 25 and the radiopaque portion 65 may be the radiopaque marker. Still alternatively, each of the radiopaque portion 25 and the radiopaque portion 65 may include a portion made of a radiopaque substance, or may be a portion formed by mixing a radiopaque substance in its material. Further alternatively, the radiopaque portion 25 may be a radiopaque marker and, additionally, the radiopaque portion 65 may either include a portion made of a radiopaque substance or be a portion formed by mixing a radiopaque substance in its material. Further alternatively, the radiopaque portion 25 may either include a portion made of a radiopaque substance or be a portion formed by mixing a radiopaque substance in its material and, additionally, the radiopaque portion 65 may be a radiopaque marker. Among these cases, preferably, the radiopaque portion 25 is a radiopaque marker and, additionally, the radiopaque portion 65 either includes a portion made of a radiopaque substance or is a portion formed by mixing a radiopaque substance in its material. Examples of the portion made of a radiopaque substance include a portion provided with a wire rod made of a radiopaque substance. Exemplary cases of including the portion made of a radiopaque substance include a case where a resin adheres to at least part of the wire rod made of a radiopaque substance. Examples of the portion formed by mixing a radiopaque substance in the material include a portion formed by mixing fine particles of the radiopaque substance in a resin.

The balloon 22 has a distance (mm) in the longitudinal axis direction of the shaft 21 from a distal end 28B of the unfixed portion 28 not fixed to the shaft 21 to the distal end 60B of the probe 60, and the distance is preferably 0.5 times or more an outer diameter (mm) of the balloon 22 provided therein with fluid at the center 22C in the longitudinal axis direction of the shaft 21. The balloon 22 being expanded and positioned closer to a terminal of the peripheral bronchus portion 101 is more likely to damage the peripheral bronchus portion 101. However, the length being 0.5 times or more can facilitate avoidance of damage due to an excessive approach of the balloon 22 to the terminal of the peripheral bronchus portion 101. Accordingly, the distance (mm) from the distal end 28B to the distal end 60B in the longitudinal axis direction of the shaft 21 is more preferably 0.7 times or more the outer diameter (mm) of the balloon 22 at the center 22C in the longitudinal axis direction, further preferably 0.9 times or more, and still further preferably 1.0 time or more. Meanwhile, the distance from the distal end 28B to the distal end 60B in the longitudinal axis direction of the shaft 21 is set to 7.0 times or less the outer diameter (mm) of the balloon 22 at the center 22C in the longitudinal axis direction, for easy expansion of a portion adjacent to the terminal of the peripheral bronchus portion 101. Accordingly, the distance from the distal end 28B to the distal end 60B in the longitudinal axis direction of the shaft 21 is preferably 7.0 times or less the outer diameter (mm) of the balloon 22 at the center 22C in the longitudinal axis direction, more preferably 5.0 times or less, and further preferably 3.0 time or less.

The medical instrument set 80 may further include a guide wire 70 provided in the second lumen 2. In an exemplary case where the guide wire 70 is disposed in the balloon catheter 20 in the second lumen 2 and the balloon catheter 20 is inserted while being guided (preceded) by the guide wire 70, the balloon catheter 20 can be easily inserted smoothly to the peripheral bronchus portion 101.

The guide wire 70 is made of a metal or the like, and examples of the metal include stainless steel and an Ni—Ti superelastic alloy. The guide wire 70 has a section being perpendicular to a longitudinal axis direction and having a shape such as a round shape or a rectangular shape. The guide wire 70 has an outer diameter preferably smaller than an outer diameter of the probe 60.

The guide wire 70 may include a radiopaque portion. The radiopaque portion of the guide wire 70 is preferably positioned distally beyond the radiopaque portion 25 of the shaft 21. This configuration facilitates grasping positional relation between the guide wire 70 and the shaft 21. Description of the radiopaque portion 25 and the radiopaque portion 65 can be referred to as to other configurations of the radiopaque portion of the guide wire 70.

The probe 60 has a distal end portion 60b preferably including a curved portion having a curvature radius of 0.1 mm or more and 10 mm or less in a sectional view in the longitudinal axis direction. The curvature radius of 0.1 mm or more facilitates avoidance of damage to the peripheral bronchus portion 101. Accordingly, the curvature radius is more preferably mm or more, and further preferably 0.4 mm or more. Meanwhile, the curvature radius of 10 mm or less facilitates insertion of the probe 60 to the peripheral bronchus portion 101. The curvature radius is thus preferably mm or less, more preferably 8 mm or less, and further preferably 6 mm or less.

The distal end portion 60b of the probe 60 is not particularly limited in terms of its shape, and examples of the shape include a columnar shape, a hemispherical shape, a spherical shape, and a tapered shape. Among these, the hemispherical shape or the spherical shape is preferred due to easy avoidance of damage to the peripheral bronchus portion 101. The distal end portion 60b having the spherical shape is exemplarily depicted in FIGS. 6 and 7, and the distal end portion 60b having the hemispherical shape is exemplarily depicted in FIG. 8. The distal end portion 60b having the tapered shape is gradually distally reduced in diameter as exemplarily depicted in FIG. 9. The probe 60 has a portion other than the distal end portion 60b, the portion having a shape such as a columnar shape, a polygonal columnar shape, or a columnar or polygonal columnar shape gradually distally reduced in diameter. Among these, the columnar shape is preferred due to easy achievement of flexibility.

As depicted in FIGS. 2, 3, and the like, preferably, the probe 60 includes a columnar portion 61 and the spherical portion 62 provided at a distal end of the columnar portion 61, and the spherical portion 62 has an outer diameter larger than an outer diameter of the columnar portion 61. The outer diameter of the spherical portion 62 larger than the outer diameter of the columnar portion 61 facilitates avoidance of damage to the peripheral bronchus portion 101 by the distal end portion 60b of the probe 60.

Preferably, the probe 60 includes the columnar portion 61 having the outer diameter (mm) smaller than an outer diameter of the shaft 21 at a proximal end 22A of the balloon 22. This configuration facilitates insertion of the probe 60 to the peripheral bronchus portion 101. Meanwhile, the outer diameter (mm) of the columnar portion 61 is preferably 0.3 times or more and 1.1 times or less the outer diameter of the shaft 21 at the distal end 22B of the balloon 22. This configuration facilitates transmission of a texture of the terminal of the peripheral bronchus portion 101. In FIG. 3, the outer diameter of the shaft 21 at the proximal end 22A corresponds to an outer diameter of an outer tube 21b of the shaft 21 at the proximal end 22A, and the outer diameter of the shaft 21 at the distal end 22B corresponds to an outer diameter of the inner tube 21a of the shaft 21 at the distal end 22B.

The probe 60 preferably contains a resin. The probe 60 containing the resin has improved flexibility. Examples of the resin include a polyamide resin, a polyester resin, a polyurethane resin, a polyolefin resin, a vinyl chloride resin, a silicone resin, and natural rubber. The probe 60 may be made of only one of these resins, or two or more of these resins. Among these resins, an elastomer resin is preferred because of its excellent flexibility. That is, the resin is preferably at least one selected from a group consisting of a polyamide elastomer, a polyester elastomer, a polyurethane elastomer, a polyolefin elastomer, a vinyl chloride elastomer, and a silicone elastomer. The radiopaque portion 65 may alternatively be obtained by molding a mixture of a radiopaque substance and the resin. The probe 60 may still alternatively be made of the resin. Specific examples of the probe 60 include resin wire, and the resin wire has an outer shape in a section in a thickness direction, examples of which include a circular shape, an elliptical shape, and a rectangular shape.

The probe 60 preferably contains a metal. The specific examples of the probe 60 include metal wire exemplarily having a distal end portion that is preferably coated with the resin or a metal coil, for easy improvement in flexibility at the distal end portion. The metal wire or the metal coil is made of a material, examples of which include a shape memory alloy such as a nickel-titanium alloy, stainless steel, titanium, a cobalt-chromium alloy, and a tungsten alloy. The stainless steel is preferred among these. The metal wire has an outer shape in a section in a thickness direction, examples of which include a circular shape, an elliptical shape, and a rectangular shape.

As depicted in FIG. 6, the probe 60 preferably has a lumen 63 having the longitudinal axis direction X. This configuration improves flexibility of the probe 60 for enhanced safety. Furthermore, a medicine such as a bronchodilator may be administered via the lumen 63.

As depicted in FIGS. 7 to 9, the probe 60 may be constituted by a solid member. The probe 60 constituted by the solid member facilitates transmission of a texture of the terminal of the peripheral bronchus portion 101.

The distal end portion 60b of the probe 60 preferably has a bending load of 0.17 N or less upon pushing the distal end portion 60b by 1.0 mm, and the bending load is obtained in accordance with a method of measuring a bending load as follows. The bending load of 0.17 N or less upon pushing the distal end portion 60b by 1.0 mm facilitates avoidance of damage to the peripheral bronchus portion 101. The bending load upon pushing the distal end portion 60b by 1.0 mm is more preferably 0.16 N or less, further preferably 0.15 N or less, and still further preferably 0.08 N or less. The bending load A₁ has a lower limit not particularly limited, but may be exemplarily 0.001 N or more.

### [Method of Measuring Bending Load]

There are prepared a lower block made of stainless steel and having a rectangular parallelepiped shape having a length in a longitudinal direction of 5 cm or more, a width perpendicular to the longitudinal direction of 2 cm or more, and a thickness of 2 cm or more, and an upper block made of stainless steel and having a rectangular parallelepiped shape having a length in a longitudinal direction of 5 cm or more, a width perpendicular to the longitudinal direction of 2 cm or more, and a thickness of 2 cm or more. The probe 60 is subsequently disposed between the lower block and the upper block such that the longitudinal axis direction of the probe 60 is parallel to the longitudinal directions of the lower block and the upper block. Subsequently, a distal end of the lower block, a distal end of the upper block, and a point distant by 3 cm in the longitudinal axis direction from the distal end 60B of the probe 60 are aligned in the longitudinal axis direction of the probe 60, and the probe 60 is fixedly sandwiched between the lower block and the upper block. Then measured is a load (N) upon pushing, by 1.0 mm in a direction perpendicular to the longitudinal axis direction of the probe 60, a portion from the distal end 60B of the probe 60 to a position distant by 1.0 mm in the longitudinal axis direction with use of a pressurizer having a rectangular pressurizing surface.

The probe 60 has a proximal end portion that may be provided with a projection or a recess for prevention of slipping and the like. The proximal end portion of the probe 60 may be or may not be fixed to the bronchoscope 50 or the like. Specifically, the proximal end portion of the probe 60 may be fixed to the sheath 5, the balloon catheter 20, or the like.

As depicted in FIG. 3, the balloon 22 being pressurized with the pressurization lumen 26 being provided with the fluid has an outer diameter (mm) at a center 22C in a longitudinal axis direction of the shaft 21, and the outer diameter is preferably larger than the outer diameter (mm) of the sheath 5 at the distal end 5B of the sheath 5. The configuration can easily make an inner diameter of peripheral bronchus 101 larger than the outer diameter of the sheath 5, for easy insertion of the sheath 5 to the peripheral bronchus 101. The outer diameter (mm) of the balloon 22 at the center 22C in the longitudinal axis direction is more preferably 1.2 times or more the outer diameter (mm) of the sheath 5 at the distal end 5B of the sheath 5, further preferably 1.5 time or more, still further preferably 2.0 times or more, and particularly preferably 2.5 times or more. Meanwhile, the outer diameter (mm) of the balloon 22 at the center 22C in the longitudinal axis direction is preferably set to be 5.0 times or less the outer diameter (mm) of the sheath 5 at the distal end 5B of the sheath 5, in order to easily avoid damage to the peripheral bronchus 101. The outer diameter is thus preferably 5.0 times or less, more preferably 4.5 times or less, further preferably 4.0 times or less, and still further preferably 3.5 times or less.

The balloon 22 is not particularly limited in terms of its shape, and can include the straight tube portion 23 and a tapered portion 24 as depicted in FIG. 3, or can include a spherical portion or a prolate spherical portion. The balloon 22 can more easily expand the peripheral bronchus portion 101 with a larger contact area with an inner wall of the peripheral bronchus portion 101. Accordingly, the balloon 22 preferably includes the straight tube portion or the prolate spherical portion, and more preferably includes the straight tube portion. Meanwhile, the balloon 22 shorter in the longitudinal axis direction can be more easily accommodated in the second lumen 2 after the balloon 22 expands and contracts, so that the balloon having the spherical portion can be easily accommodated.

In the case where the balloon 22 includes the straight tube portion 23 and is pressurized with the pressurization lumen 26 being provided with the fluid, the straight tube portion 23 has a length (mm) from a proximal end 23A to a distal end 23B in the longitudinal axis direction of the shaft 21, and the length is preferably equal to or more than the outer diameter (mm) of the balloon 22 at the center 22C in the longitudinal axis direction of the shaft 21. This facilitates wide expansion of the peripheral bronchus 101, for easy insertion of the sheath 5 to the peripheral bronchus 101. Accordingly, the length (mm) from the proximal end 23A to the distal end 23B is preferably 1.1 times or more the outer diameter (mm) of the balloon 22 at the center 22C in the longitudinal axis direction, and more preferably 1.2 times or more. Meanwhile, the length (mm) from the proximal end 23A to the distal end 23B has an upper limit not particularly limited, and can be exemplarily five times or less the outer diameter (mm) of the balloon 22 at the center 22C in the longitudinal axis direction.

In the case where the balloon 22 includes the spherical portion or the prolate spherical portion, the spherical portion or the prolate spherical portion has a length (mm) from a proximal end to a distal end in the longitudinal axis direction of the shaft 21, and the length is preferably equal to or more than the outer diameter (mm) of the balloon 22 at the center 22C in the longitudinal axis direction of the shaft 21. This facilitates wide expansion of the peripheral bronchus 101, for easy insertion of the sheath 5 to the peripheral bronchus 101. Accordingly, the length (mm) from the proximal end to the distal end is more preferably 1.1 times or more the outer diameter (mm) of the balloon 22 at the center 22C in the longitudinal axis direction, and further preferably 1.2 times or more. Meanwhile, the length (mm) from the proximal end to the distal end has an upper limit not particularly limited, and can be exemplarily five times or less the outer diameter (mm) of the balloon 22 at the center 22C in the longitudinal axis direction.

The balloon 22 is preferably a noncompliant balloon or a semicompliant balloon. These balloons are each hard to be expanded when fluid injection pressure has a predetermined value or more. This can facilitate avoidance of damage to the peripheral bronchus 101 due to excessive expansion of the balloon 22. The semicompliant balloon is more preferred because the semicompliant balloon easily achieves both safety and expansion of the peripheral bronchus 101. The noncompliant balloon has 4% or less as an outer diameter change rate of the balloon 22 at the center 22C in the longitudinal axis direction upon pressurization from 50% of specified pressure (pressure having a nominal value=nominal pressure) of the balloon to the specified pressure. In contrast, the semicompliant balloon has more than 4% and 15% or less as the outer diameter change rate of the balloon 22 at the center 22C in the longitudinal axis direction upon pressurization from 50% of specified pressure (pressure having a nominal value=nominal pressure) of the balloon to the specified pressure. The specified pressure is preferably 2 atm or more and 20 atm or less, and more preferably 3 atm or more and 10 atm or less.

The balloon 22 may be made of a resin, examples of which include a polyamide resin, a polyester resin, a polyurethane resin, a polyolefin resin, a vinyl chloride resin, a silicone resin, and natural rubber. The balloon 22 may be made of only one of these resins, or two or more of these resins.

The balloon 22 may alternatively include a reinforcing material provided on an outer side surface of a resin layer made of the resin, for improvement in dimensional safety against expansion pressure. Examples of the reinforcing material include a fiber material. The reinforcing material may be provided entirely on the outer side surface of the resin layer without any gap, or may be provided only partially on the outer side surface of the resin layer. Examples of the fiber material adopted as the reinforcing material include polyarylate fiber, aramid fiber, ultrahigh molecular weight polyethylene fiber, PBO fiber, and carbon fiber. Each of these fiber materials may be a monofilament or a multifilament.

The fluid injected to the pressurization lumen 26 of the balloon 22 is not particularly limited, and can be liquid or gas. The fluid may be pressurized with use of a syringe, an indeflator, a pump, or the like before being injected to the pressurization lumen 26.

The number the balloon 22 is not limited to one, and there may be two or more balloons. The balloon 22 smaller in the number more easily enters and leaves the second lumen 2, so that there is preferably provided one balloon 22.

In a section perpendicular to the longitudinal axis direction of the sheath 5, the second lumen 2 preferably has an area (mm²) being 1.2 times or more an area (mm²) of a region surrounded with an outer circumferential line of the balloon 22 being contracted. Also in the state where the balloon 22 is disposed in the second lumen 2, negative pressure can thus be applied to the second lumen 2 to easily stick a distal end 5B of the sheath 5 to an inner wall of the bronchus 100. Furthermore, the distal end 5B of the sheath 5 sticking to the inner wall of the bronchus 100 is slid to wipe an impurity in the bronchus 100 adhering to an endoscopic camera 10. Accordingly, the area (mm²) of the second lumen 2 is more preferably 1.3 times or more the area (mm²) of the region surrounded with the outer circumferential line of the balloon 22 being contracted, and further preferably 1.4 times or more. Meanwhile, the area (mm²) of the second lumen 2 is eight times or less the area (mm²) of the region surrounded with the outer circumferential line of the balloon 22 being contracted, for easy avoidance of damage due to excessive sticking of the distal end 5B of the sheath 5 to the inner wall of the bronchus 100. The area is more preferably 6.5 times or less, and further preferably 5 times or less.

As depicted in FIGS. 2, 3, and 5, the shaft 21 may include the inner tube 21a and the outer tube 21b. The inner tube 21a can be utilized as an insertion path for guide wire or the like. In FIGS. 2, 3, and 5, the balloon 22 has a distal end portion fixed to the inner tube 21a at the distal fixed portion 27, and a proximal end portion fixed to the outer tube 21b at the proximal fixed portion 27. Such a configuration can utilize a space between the inner tube 21a and the outer tube 21b as a flow path for fluid injection, and can further utilize a space between the inner tube 21a and the balloon 22 as the pressurization lumen 26. The inner tube 21a has a distal end 21B that may be positioned distally beyond the distal end 22B of the balloon 22 as depicted in FIGS. 3 and 5, or may be aligned with the distal end 22B in the longitudinal axis direction.

As depicted in FIG. 10, the shaft 21 may include the outer tube 21b and a linear body 21c disposed in the outer tube 21b. Accordingly, the distal end portion of the balloon 22 can be fixed to the linear body 21c at the distal fixed portion 27, and the proximal end portion of the balloon 22 can be fixed to the outer tube 21b at the proximal fixed portion 27. Such a configuration can utilize a space between the linear body 21c and the outer tube 21b as the flow path for fluid injection, and can further utilize a space between the linear body 21c and the balloon 22 as the pressurization lumen 26. The linear body 21c has a distal end 21C that may be positioned distally beyond the distal end 22B of the balloon 22 as depicted in FIG. 10, or may be aligned with the distal end 22B in the longitudinal axis direction.

Though not depicted, the shaft 21 may alternatively be constituted by the linear body 21c without including the inner tube 21a or the outer tube 21b.

The inner tube 21a may contain at least one selected from a group consisting of a polyamide resin, a polyester resin, a polyurethane resin, a polyolefin resin, a fluororesin, a vinyl chloride resin, a silicone resin, and natural rubber, or may contain at least one selected from a group consisting of a polyamide elastomer, a polyester elastomer, a polyurethane elastomer, a polyolefin elastomer, a vinyl chloride elastomer, and a silicone elastomer. The inner tube 21a may be made of only one of these materials, or two or more of these materials. The inner tube 21a preferably contains at least one selected from a group consisting of the polyamide resin, the polyolefin resin, and the fluororesin.

The inner tube 21a may optionally include a reinforcing member. Examples of the reinforcing member include a braided body made of a braided wire rod, and a coil body made of a spirally wound wire rod. This facilitates improvement in strength of the inner tube 21a.

Examples of the wire rod constituting the reinforcing member include metal wire and fiber. The metal wire is preferably made of a material such as stainless steel, titanium, a nickel-titanium alloy, a cobalt-chromium alloy, and a tungsten alloy. Among these, the stainless steel is more preferred. The metal wire may include a single wire or a twisted wire. Examples of the fiber include polyarylate fiber, aramid fiber, ultrahigh molecular weight polyethylene fiber, PBO fiber, and carbon fiber. The fiber may be a monofilament or a multifilament.

The outer tube 21b may contain at least one selected from a group consisting of a polyamide resin, a polyester resin, a polyurethane resin, a polyolefin resin, a fluororesin, a vinyl chloride resin, a silicone resin, and natural rubber, or may contain at least one selected from a group consisting of a polyamide elastomer, a polyester elastomer, a polyurethane elastomer, a polyolefin elastomer, a vinyl chloride elastomer, and a silicone elastomer. The outer tube 21b may be made of only one of these materials, or two or more of these materials. The outer tube 21b preferably contains at least one selected from a group consisting of the polyamide resin, the polyolefin resin, and the polyurethane resin.

Examples of the linear body 21c include metal wire and resin wire. The metal wire has a distal end portion that is preferably coated with a resin (a so-called polymer jacket type) or a metal coil (a so-called coil jacket type), for easy improvement in flexibility at the distal end portion. The metal wire or the resin wire has an outer shape in a section in a thickness direction, examples of which include a circular shape, an elliptical shape, and a rectangular shape.

The metal wire or the metal coil is made of a material, examples of which include a shape memory alloy such as a nickel-titanium alloy, stainless steel, titanium, a cobalt-chromium alloy, and a tungsten alloy. The stainless steel is preferred among these.

Examples of the resin covering the resin wire or the metal wire include a polyamide resin, a polyester resin, a polyurethane resin, a polyolefin resin, a fluororesin, a vinyl chloride resin, a silicone resin, and natural rubber. The resin is preferably at least one selected from a group consisting of a polyamide elastomer, a polyester elastomer, a polyurethane elastomer, a polyolefin elastomer, a vinyl chloride elastomer, and a silicone elastomer.

The balloon catheter 20 preferably includes a bar portion 59 extending distally from the distal end 28B of the unfixed portion 28 as depicted in FIG. 10.

The balloon catheter 20 preferably has a bending load of 0.17 N or less upon pushing the bar portion 59 by 1.0 mm, and the bending load is obtained in accordance with a method of measuring a bending load as follows. The bending load of 0.17 N or less upon pushing the bar portion 59 by 1.0 mm facilitates avoidance of damage to the peripheral bronchus portion 101. The bending load upon pushing the bar portion 59 by 1.0 mm is more preferably 0.15 N or less, further preferably 0.12 N or less, and still further preferably 0.08 N or less. The bending load has a lower limit not particularly limited, but may be exemplarily 0.003 N or more.

### [Method of Measuring Bending Load]

There are prepared a lower block made of stainless steel and having a rectangular parallelepiped shape having a length in a longitudinal direction of 5 cm or more, a width perpendicular to the longitudinal direction of 2 cm or more, and a thickness of 2 cm or more, and an upper block made of stainless steel and having a rectangular parallelepiped shape having a length in a longitudinal direction of 5 cm or more, a width perpendicular to the longitudinal direction of 2 cm or more, and a thickness of 2 cm or more. The balloon catheter 20 is subsequently disposed between the lower block and the upper block such that the longitudinal axis direction of the shaft 21 is parallel to the longitudinal directions of the lower block and the upper block. Subsequently, a distal end of the lower block, a distal end of the upper block, and the distal end 28B of the unfixed portion 28 of the balloon 22 are aligned in the longitudinal axis direction of the shaft 21, and the balloon catheter 20 is fixedly sandwiched between the lower block and the upper block. Then measured is a load (N) upon pushing, by 1.0 mm in a direction perpendicular to the longitudinal axis direction of the shaft 21, a portion from a distal end of the bar portion 59 to a position distant by 1.0 mm in the longitudinal axis direction of the shaft 21 with use of a pressurizer having a rectangular pressurizing surface.

As depicted in FIG. 1, the balloon catheter 20 has a proximal portion preferably provided with a handle 29. The handle 29 preferably has a lumen communicating with the inner tube 21a and extending in a longitudinal axis direction. The lumen can be utilized as an insertion path for guide wire or the like. Preferably, the handle 29 further includes a fluid injection portion 29a and has a lumen communicating with a flow path for fluid injection to the pressurization lumen 26 of the balloon 22.

The second lumen 2 has a proximal end portion preferably coupled with a negative pressure generator. The negative pressure generator exemplarily includes a pump. The negative pressure generator (not depicted) may be attached to a negative pressure generator attachment port 51 communicating directly or indirectly to a proximal end of the second lumen 2 of the sheath 5, as exemplarily depicted in FIG. 1.

As depicted in FIG. 11, at the distal end 5B of the sheath 5, a distance between a center of the second lumen 2 and a center of the object lens 11 is preferably two times or less a diameter of the second lumen 2. This disposition facilitates removal of an impurity adhering to the object lens 11 upon sliding the distal end 5B of the sheath 5 sticking to the inner wall of the bronchus 100. The distance is more preferably 1.5 times or less, and further preferably 1.2 times or less. The distance has a lower limit that may be exemplarily 0.6 times or more.

As depicted in FIG. 11, at the distal end 5B of the sheath 5, the center of the second lumen 2 is preferably not positioned at a center of the sheath 5. This disposition allows a distal end 2B of the second lumen 2 to easily stick to a desired position by rotation or the like of the sheath 5 in the longitudinal axis direction.

At the distal end 5B of the sheath 5, the diameter (mm) of the second lumen 2 is preferably equal to or more than a diameter (mm) of the object lens 11. This configuration allows the distal end 5B of the sheath 5 to easily stick to the inner wall of the bronchus 100, as well as facilitates removal of an impurity adhering to the object lens 11. The diameter (mm) of the second lumen 2 is more preferably 1.2 times or more the diameter (mm) of the object lens 11, and further preferably 1.5 times or more. Meanwhile, the diameter (mm) of the second lumen 2 may be 3.0 times or less the diameter (mm) of the object lens 11, or may be 2.5 times or less.

At the distal end 5B of the sheath 5, assuming that an area of a region surrounded with an outer circumference of the sheath 5 has 100 area percent, the second lumen 2 has an area ratio that is preferably 10 area percent or more. This configuration improves sticking force and allows the distal end 5B of the sheath 5 to easily stick to the inner wall of the bronchus 100. The area ratio is more preferably 15 area percent or more, and further preferably 20 area percent or more. Meanwhile, the area ratio has an upper limit not particularly limited, and may be 80 area percent or less, or may be 60 area percent or less.

In the longitudinal axis direction of the sheath 5, the object lens 11 has a distal end preferably positioned identically with the distal end 5B of the sheath 5 as depicted in FIG. 2 or positioned distally to the distal end 5B of the sheath 5. This disposition facilitates wiping an impurity in the bronchus 100 adhering to the object lens 11. A distance between the distal end of the object lens 11 and the distal end 5B of the sheath 5 in the longitudinal axis direction of the sheath 5 is preferably 5 mm or less, more preferably 3 mm or less, and further preferably 1 mm or less. This disposition allows the distal end 5B of the sheath 5 to easily stick to the inner wall of the bronchus 100.

As depicted in FIG. 11, the sheath 5 may have a third lumen 3 having the longitudinal axis direction X, and may further have a fourth lumen 4 having the longitudinal axis direction X. The third lumen 3 may be provided therein with a first illumination lens 30, and the fourth lumen 4 may be provided therein with a second illumination lens 40. This facilitates observation of the peripheral bronchus 101. The first illumination lens 30 and the second illumination lens 40 are preferably fixed to the third lumen 3 and the fourth lumen 4, respectively, so as not to move in the longitudinal axis direction X.

The lumens other than the second lumen 2 are preferably sealed at the distal end 5B of the sheath 5. This facilitates improvement in sticking force in the second lumen 2.

The sheath 5 has an outer side surface preferably provided with no through hole communicating with the second lumen 2 from the distal end 5B of the sheath 5 to a position distant by 1 cm in the longitudinal axis direction. This facilitates improvement in sticking force of the second lumen 2. Furthermore, the outer side surface of the sheath 5 from the distal end 5B of the sheath 5 to a position distant by 3 cm in the longitudinal axis direction is more preferably provided with no through hole communicating with the second lumen 2, and the outer side surface of the sheath 5 from the distal end 5B to a proximal end of the sheath 5 is further preferably provided with no through hole.

As depicted in FIG. 1, the sheath 5 has a proximal portion preferably provided with an operation portion 55 incorporating the proximal portion of the sheath 5. The operation portion 55 thus provided allows an operator to grip the operation portion 55 for adjustment of an insertion angle or the like of the sheath 5. Examples of the operation portion 55 include a resin case.

The proximal portion of the sheath 5 may be optionally divided into two or more branches. In an exemplary case where the proximal portion of the sheath 5 is divided into two branches, the proximal end of the second lumen 2 at a first one of the branches may be coupled to the negative pressure generator attachment port 51 at the operation portion 55, and the proximal end of the second lumen 2 at a second one of the branches may be coupled to the insertion hole 52 at the operation portion 55. Alternatively, the proximal portion of the sheath 5 may not be branched. In this case, the proximal end of the second lumen 2 may be coupled to the negative pressure generator attachment port 51, and there may be provided a through hole from the second lumen 2 to the outer side surface so as to communicate with a passage reaching the insertion hole 52 in the operation portion 55. Alternatively, the proximal end of the second lumen 2 may be coupled to the insertion hole 52, and there may be provided a through hole from the second lumen 2 to the outer side surface so as to communicate with a passage reaching the negative pressure generator attachment port 51 in the operation portion 55.

An insertion hole 52 is preferably provided with a sealing member configured to seal the insertion hole 52 when negative pressure is applied to the second lumen 2. This facilitates improvement in sticking force of the second lumen 2. The insertion hole 52 is preferably provided with a sealing member configured to seal the insertion hole 52 when the negative pressure N is applied to the second lumen 2. This facilitates improvement in sticking force of the second lumen 2. Examples of the sealing member include a forceps plug provided with a cut, and specifically include a silicon rod having a cut for a Y connector.

The present invention further includes a method of using the medical instrument set 80. The method of using the medical instrument set 80 includes inserting the probe 60 to the second lumen 2, extruding the distal end portion 60b of the probe 60 from a distal end 2B of the second lumen 2, and then extruding the balloon 22 from the distal end 2B of the second lumen 2. The probe 60 preceding the balloon catheter 20 detects the peripheral bronchus portion 101, to facilitate avoidance of damage to a visceral pleura due to forcible and excessive insertion of the balloon catheter 20.

Preferably, the method of using the medical instrument set 80 further includes applying positive pressure to the balloon 22 to expand the balloon 22, applying negative pressure to the balloon 22 to contract the balloon 22, pulling back the balloon 22 into the second lumen 2, distally pushing the bronchoscope 50, and observing with use of the endoscopic camera 10. When the bronchoscope 50 is inserted to the peripheral bronchus portion 101 in a state where the peripheral bronchus portion 101 is expanded by the balloon 22 and the balloon 22 is contracted, insertion resistance is reduced for easy insertion.

The balloon 22 may be expanded in a state where the probe 60 is extended from the second lumen 2 as depicted in FIG. 3, or after the probe 60 is pulled back into the second lumen 2.

When applying positive pressure to the balloon 22 to expand the balloon 22, a distance (mm) from the distal end 5B of the sheath 5 to the proximal end 28A of the unfixed portion 28 of the balloon 22 upon application of the positive pressure is preferably three times or less a distance (mm) from the proximal end 28A to the distal end 28B of the unfixed portion 28 of the balloon 22. This disposition facilitates insertion before contraction of the peripheral bronchus portion 101 thus expanded.

The method of using the medical instrument set 80 may include applying negative pressure to the second lumen 2 to cause the distal end 5B of the sheath 5 to stick to the inner wall of the bronchus 100. The method may further include sliding the distal end 5B of the sheath 5 sticking to the inner wall of the bronchus 100, to remove an impurity in the bronchus 100 adhering to a distal end of the endoscopic camera 10. This facilitates observation of the peripheral bronchus portion 101.

The present application claims benefit of priority based on Japanese Patent application No. 2019-189684 filed on Oct. 16, 2019. The entire contents of the specification of Japanese Patent application No. 2019-189684 filed on Oct. 16, 2019 are incorporated in the present application for reference.

### DESCRIPTION OF REFERENCE SIGNS

1 first lumen
2 second lumen
2B distal end of second lumen
3 third lumen
4 fourth lumen
5 sheath
5B distal end of sheath
10 endoscopic camera
11 object lens
12 image transmitter
20 balloon catheter
21 shaft
21a inner tube
21b outer tube
21c linear body
21C distal end of linear body
22 balloon
22A proximal end of balloon
22B distal end of balloon
22C center of balloon in longitudinal axis direction of shaft
23 straight tube portion
23A proximal end of straight tube portion
23B distal end of straight tube portion
24 tapered portion
25 radiopaque portion
26 pressurization lumen
27 fixed portion
28 unfixed portion
28A proximal end of unfixed portion
28B distal end of unfixed portion
29 handle
29a injection portion
30 first illumination lens
40 second illumination lens
50 bronchoscope
51 negative pressure generator attachment port
52 insertion hole
53 connector portion
55 operation portion
59 bar portion
60 probe
60B distal end of probe
60b distal end portion of probe
61 columnar portion
62 spherical portion
63 lumen of probe
65 radiopaque portion
70 guide wire
80 medical instrument set
100 bronchus
101 peripheral bronchus portion

1. A medical instrument set comprising:
a bronchoscope including
a sheath having a longitudinal axis direction, the sheath having a first lumen and a second lumen each extending in the longitudinal axis direction,
an endoscopic camera disposed in the first lumen, and
a balloon catheter including a shaft and a balloon provided at a distal portion of the shaft, the balloon catheter being disposed in the second lumen so that the balloon catheter is movable in the longitudinal axis direction; and
a probe for detecting a peripheral bronchus portion, the bronchoscope and the probe being configured so that the probe has a distal end positioned distally beyond a distal end of the balloon.

2. The medical instrument set according to claim 1, wherein the probe is inserted to the second lumen of the bronchoscope.

3. The medical instrument set according to claim 1, wherein the shaft and the probe each include a radiopaque portion.

4. The medical instrument set according to claim 3, wherein the bronchoscope and the probe are configured so that the radiopaque portion of the probe is positioned distally beyond the radiopaque portion of the shaft.

5. The medical instrument set according to claim 3, wherein the shaft includes the radiopaque portion positioned proximally beyond a proximal end of an unfixed portion of the balloon, the unfixed portion not being fixed to the shaft.

6. The medical instrument set according to claim 3, wherein the radiopaque portion is a radiopaque marker.

7. The medical instrument set according to claim 1, wherein the balloon has a distance (mm) in a longitudinal axis direction of the shaft from a distal end of an unfixed portion not fixed to the shaft to the distal end of the probe, the distance being 0.5 times or more an outer diameter (mm) of the balloon provided therein with fluid at a center in the longitudinal axis direction of the shaft.

8. The medical instrument set according to claim 1, further comprising a guide wire provided in the second lumen.

9. The medical instrument set according to claim 1, wherein the probe has a distal end portion including a curved portion having a curvature radius of 0.1 mm or more and 10 mm or less in a sectional view in a longitudinal axis direction.

10. The medical instrument set according to claim 1, wherein the probe contains comprises a resin.

11. The medical instrument set according to claim 1, wherein the probe contains comprises a metal.

12. The medical instrument set according to claim 1, wherein the probe has a lumen extending in a longitudinal axis direction.

13. The medical instrument set according to claim 1, wherein the probe includes a columnar portion having an outer diameter (mm) smaller than an outer diameter of the shaft at a proximal end of the balloon.

14. The medical instrument set according to claim 1, wherein the probe includes a columnar portion and a spherical portion provided at a distal end of the columnar portion, and the spherical portion has an outer diameter larger than an outer diameter of the columnar portion.

15. A method of using the medical instrument set according to claim 1, the method comprising:
inserting the probe to the second lumen of the sheath;
extruding a distal end portion of the probe from a distal end of the second lumen; and then
extruding the balloon from the distal end of the second lumen.
